(19) United States
(12) Patent Application Publication
(10) Pub. No.: US 2025/0288316 A1
(43) Pub. Date: Sep. 18, 2025

(54) SURGICAL SAFETY KNIFE

(71) Applicant: ACCUTEC, INC., Verona, VA (US)

(72) Inventors: Lucas Aaron Duvall, Harrisonburg, VA (US); Manuel Lamazares, Miami Shores, FL (US); Adam Charles Magoun, Eliot, ME (US); Peter Chaikowsky, Waynesboro, VA (US)

(21) Appl. No.: 18/861,014

(22) PCT Filed: May 1, 2023
(86) PCT No.: PCT/US2023/020631

§ 371 Date: Oct. 28, 2024

Related U.S. Application Data
(60) Provisional application No. 63/336,653, filed on Apr. 29, 2022.

(51) Int. Cl. A61B 17/3211

(52) U.S. Cl. CPC A61B 17/3211; A61B 2017/32113

(57) ABSTRACT

In one aspect, a surgical safety knife is provided with an elongated handle with first and second notches, spaced apart along the length thereof; a blade extending distally from the distal portion; and, a shield moveable along the handle between a first position, where the blade is exposed, to a second position, where the shield fully covers the blade, wherein, the shield includes a main body and a lever pivotally coupled to the main body, wherein, a tab is provided on the lever formed to be selectively snap received within the first notch and the second notch with the lever being in an undisplaced state, wherein, with the tab being received in the first notch, the shield is retained in the first position, and, wherein, displacement of the lever to the displaced state results in the tab being freed from the first notch to release the shield.

### CROSS-REFERENCE TO RELATED APPLICATION

This application claims priority to U.S. Provisional Appl. No. 63/336,653, filed Apr. 29, 2022, the entire contents of which are incorporated by reference herein.

### BACKGROUND OF THE INVENTION

Surgical safety knives are well known in the prior art. These designs typically include a surgical blade affixed to a handle with a shield, advanceable to cover the blade post-use.

Typical designs cause the shield to lock in a safety position fully covering the surgical blade. To avoid inadvertent or preventable shielding of the blade, different arrangements have been provided to retain the shield in a use state, with the blade being exposed. Two hands may be needed to advance the shield from the use state to the safety position. Moreover, release features may be located in the grip area of the handle, requiring a surgeon to manipulate the handle about these features.

### SUMMARY OF THE INVENTION

In one aspect, a surgical safety knife is provided with an elongated handle having a proximal portion, defining a grip for a user, and a distal portion, having a reduced cross-section compared to the proximal portion, wherein, the distal portion is elongated with first and second notches being formed thereon, spaced apart along the length of the distal portion; a blade extending distally from the distal portion of the handle; and, a shield moveably mounted to the distal portion of the handle so as to be moveable along the handle between a first position, where the blade is exposed, to a second position, where the shield fully covers the blade, wherein, the shield includes a main body and a lever pivotally coupled to the main body, the main body being in contact with the distal portion of the handle, the lever being pivotally connected to the main body so as to be pivotally displaceable relative to the main body between an undisplaced state and a displaced state, wherein, a tab is provided on the lever formed to be selectively snap received within the first notch and the second notch with the lever being in the undisplaced state, wherein, with the tab being received in the first notch, the shield is retained in the first position, and, wherein, displacement of the lever to the displaced state results in the tab being freed from the first notch to release the shield and to allow the shield to move along the handle from the first position.

In a further aspect, a surgical safety knife is provided with an elongated handle having a proximal portion, defining a grip for a user, and a distal portion, having a reduced cross-section compared to the proximal portion, wherein, the distal portion is elongated with first and second tabs protruding therefrom, spaced apart along the length of the distal portion; a blade extending distally from the distal portion of the handle; and, a shield moveably mounted to the distal portion of the handle so as to be moveable along the handle between a first position, where the blade is exposed, to a second position, where the shield fully covers the blade, wherein, the shield includes a main body and a lever pivotally coupled to the main body, the main body being in contact with the distal portion of the handle, the lever being pivotally connected to the main body so as to be pivotally displaceable relative to the main body between an undisplaced state and a displaced state, wherein, at least one notch is provided on the lever formed to selectively snap receive one of the first tab and the second tab with the lever being in the undisplaced state, wherein, with the first tab being received in the at least one notch, the shield is retained in the first position, and, wherein, displacement of the lever to the displaced state results in the first tab being freed from the at least one notch to release the shield and to allow the shield to move along the handle from the first position.

Advantageously, the subject invention provides a surgical safety knife allowing for one-hand movement of the shield with the shield release feature being separate from the grip portion of the handle.

As used herein, the term “distal,” and derivatives thereof, shall refer to a direction towards a patient (away from the user), whereas, the term “proximal,” and derivatives thereof, shall refer to a direction towards a user (away from the patient).

These and other features of the invention will be better understood through a study of the following detailed description and accompanying drawings.

### BRIEF DESCRIPTION OF THE DRAWINGS

FIG. 1 is a perspective view of a surgical safety knife in accordance with the subject invention having a shield in a first, use position with the blade being fully exposed;

FIG. 2 is a side view of the surgical safety knife shown in FIG. 1;

FIG. 3 is a bottom view of the surgical safety knife shown in FIG. 1;

FIG. 4 is a perspective view of the surgical safety knife shown in FIG. 1 having the shield in a second, post-use position with the shield fully covering the blade;

FIG. 5 is a side view of the surgical safety knife shown in FIG. 4;

FIG. 6 is a bottom view of the surgical safety knife shown in FIG. 4;

FIGS. 7-9 show interaction between a tab formed on the lever of the shield and notches formed on a distal portion of the handle of a surgical safety knife formed in accordance with the subject invention;

FIGS. 10-13 are different views of the surgical safety knife of FIG. 1 with the shield in different positions providing limited exposure of the blade;

FIG. 14 is an end view of the surgical safety knife of FIG. 1;

FIGS. 15-18 are different views of an alternate surgical safety knife formed in accordance with the subject invention showing the shield in a use state (blade exposed) and post-use state (blade covered);

FIGS. 19A-19F show alternate embodiments of a surgical safety knife in accordance with the subject invention with a two-step release of the shield and a modified one-step release of the shield;

FIG. 20 shows an alternative configuration of the notches useable with the subject invention;

FIGS. 21-24 show a delimited range of motion of the shield relative to the handle, with FIG. 22 be a cross-sectional view of FIG. 21 taken along line A-A, FIG. 23 being a similar view to FIG. 22 with the position of the shield adjusted, and, FIG. 24 being an enlarged view of Section B in FIG. 23;

FIGS. 25-28 show an alternate embodiment of a surgical safety knife in accordance with the subject invention with the tabs located on the handle and the notches located on the shield; and,

FIGS. 29-33 show alternate versions of the embodiment of FIGS. 25-28 where the tabs are pin shaped.

### DETAILED DESCRIPTION OF THE INVENTION

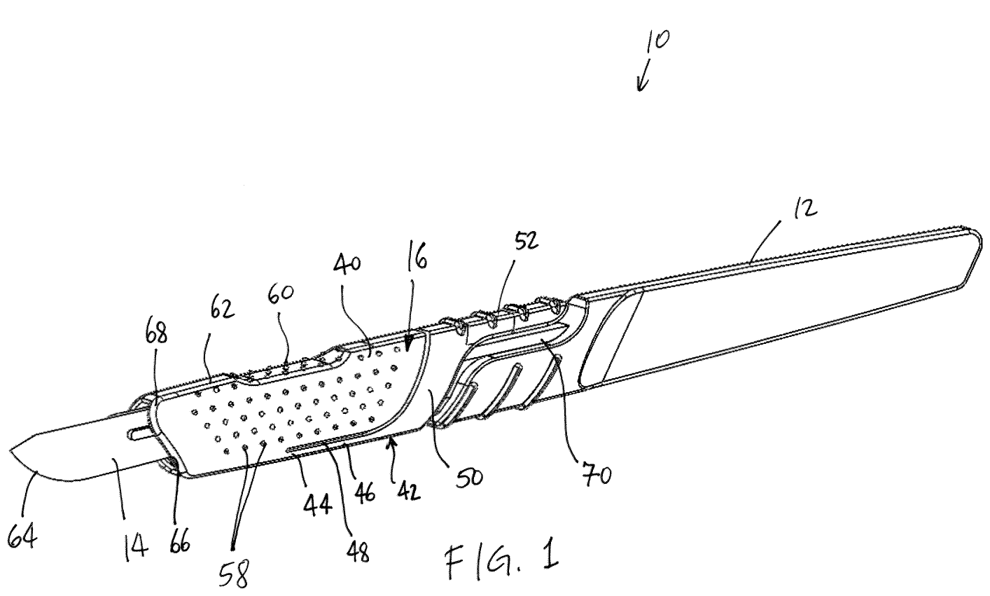

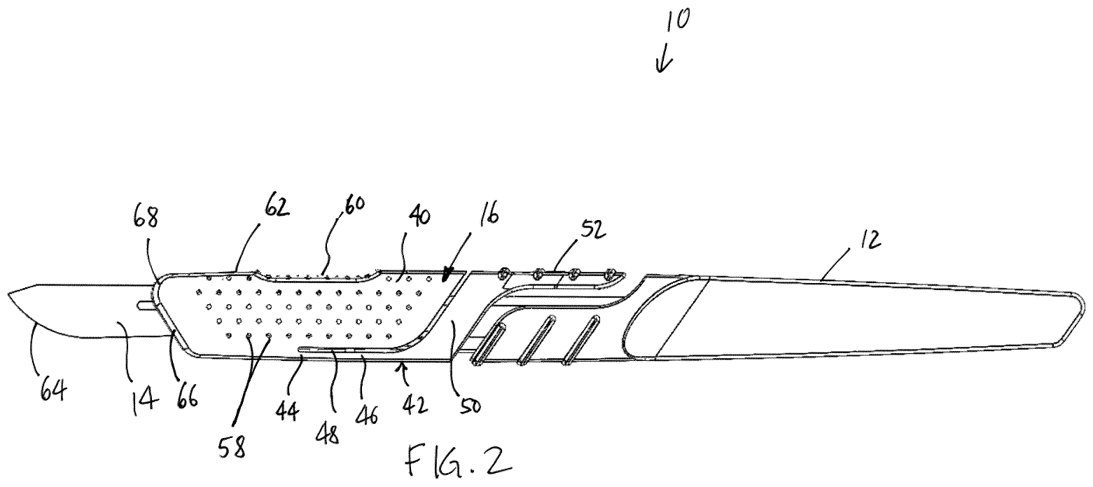

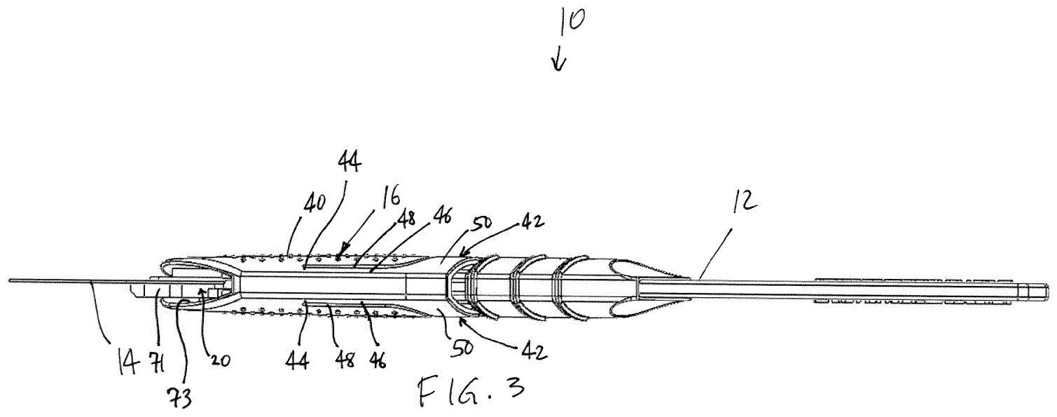

As shown in the Figures, a surgical safety knife is shown and generally designated with reference number 10. The surgical safety knife 10 generally includes a handle 12, a blade 14 mounted to the handle 12, and a shield 16 movably mounted to the handle 12. The shield 16 is movable along the length of the handle 12 between a first position, where the blade 14 is exposed, e.g., as shown in FIGS. 1-3, to a second position, where the shield 16 fully covers the blade 14, e.g., as shown in FIGS. 4-6. The subject invention allows for shielding of a used blade post-use to prevent inadvertent contact therewith.

The handle 12 generally includes a proximal portion 18 and a distal portion 20. The proximal portion 18 defines a grip for a user. The grip may be contoured with one or more grip enhancing features 22, such as ribs, ridges, raised dots, indentations, knurled surfacing, textured surfacing, and so forth, aligned for engagement by a user's thumb and/or forefinger. The proximal portion 18 may be formed of, or coated with, a metallic material and/or thermoplastic material and/or elastomeric material, preferably, which is sterilizable. The proximal portion 18 may have a generally board-shaped rear (promixal) portion 18A with a wider forward (distal) portion 18B. The forward portion 18B may define, along at least an axial portion thereof, an oval profile, with the top and bottom thereof being optionally flattened.

The distal portion 20 extends distally from the proximal portion 18 and may be provided with a reduced cross-section compared to adjacent portions of the proximal portion 18. The distal portion 20 is elongated and may be provided with a generally rectangular cross-section having opposing first and second lateral faces 24, 26, each with width W1, and opposing upper and lower edges 28, 30, the upper edge 28 having width W2, and the lower edge 30 having width W3. The widths W2 and W3, which may be the same, are preferably less than the width W1.

At least one channel 32 is formed in the distal portion 20, e.g., along at least a portion of the length of the first lateral face 24. Preferably, two of the channels 32 are provided, aligned in parallel along the respective lengths of the first and second lateral faces 26. A guide rib 34 may be provided along the length of each of the channels 32. The distal portion 20 may be also provided with an enlarged cross-section along the upper edge 28 to define a rail 36. The rail 36 may have a generally rectangular cross-section. With the enlarged cross-section, the width W2 of the upper edge 28 is greater than the width W3 of the lower edge 30.

A plurality of notches 38 are formed along the length of the distal portion 20, particularly to be spaced apart. The notches 38 are preferably formed along the lower edge 30, with the notches 38 being similarly shaped and dimensioned and facing in the same orientation. The notches 38 preferably extend across the full width W3 of the lower edge 30 and face downwardly.

The distal portion 20 may be formed from metallic material and/or thermoplastic material, with the distal portion 20 being joined to the proximal portion 18 using any known technique, e.g., adhesion, fusion, interference fit, co-injection molding, and so forth. Optionally, the distal portion 20 may be formed integrally with the proximal portion 18.

The shield 16 is tubular with the distal portion 20 formed to extend therethrough. The shield 16 generally includes a main body 40 and a lever 42. The lever 42 is pivotally connected to the main body 40 by pivotal connection 44. The pivotal connection 44 acts as a hinge in allowing the lever 42 to be pivotally displaced relative to the main body 40 between an undisplaced state (i.e., an at-rest state) and a displaced state. By way of non-limiting example, the lever 42 may be formed to include a cantilevered member 46 extending from the pivotal connection 44. The pivotal connection 44 may be formed by providing at least one relief slot 48 separating the main body 40 and the lever 42. Preferably, the pivotal connection 44 is formed with inherent memory which urges the lever 42 to the undisplaced state. The lever 42 may be displaced by applying downward force on the lever 42 thereby causing the lever 42 to pivot relative to the main body 40, about the pivotal connection 44. The downward force causes a moment to be generated about the pivotal connection 44, resulting in downward angular displacement of the cantilevered member 46. A yoke 50 may be provided with the lever 42 formed to extend upwardly away from the cantilevered member 46 to provide an exposed upward-facing surface for engagement by a user. Further, a tail 52 may be provided with the lever 42 formed to extend from the yoke 50, e.g., extend proximally. The tail 52 may act as a lever arm which advantageously provides mechanical advantage to any force applied thereto. With this arrangement, downward force applied to the yoke 50 and/or the tail 52 may result in displacement of the lever 42. With removal of any displacing force, restoring force generated by the inherent memory of the pivotal connection 44 will urge the lever 42 to return towards the undisplaced state.

Inherent memory may be formed in the pivotal connection 44 through any known technique. For example, the shield 16 may be formed from a resilient thermoplastic having sufficient resiliency to avoid inelastic deformation with the lever 42 being displaced between the undisplaced state and the displaced state. Material treatment may be additionally, or alternatively, utilized, such as tempering or the like.

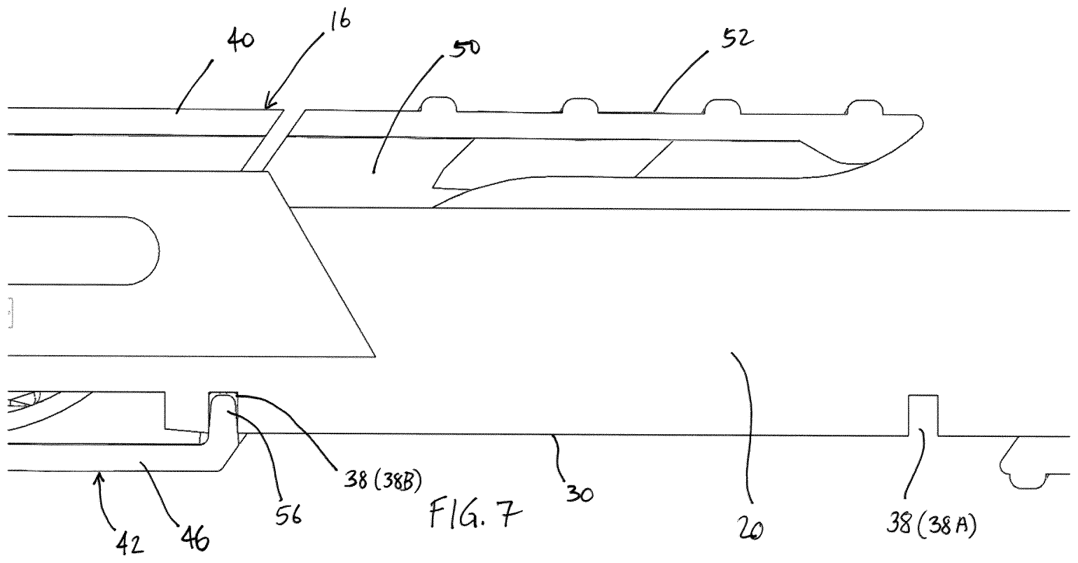

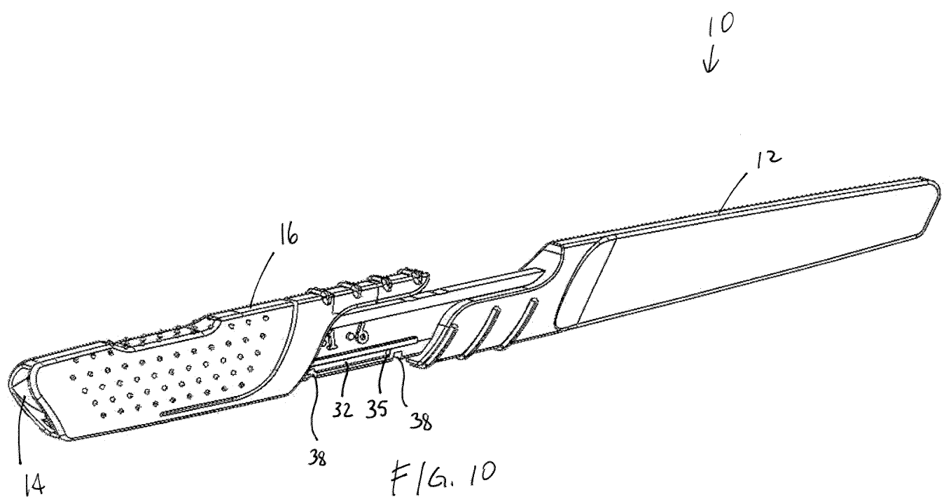

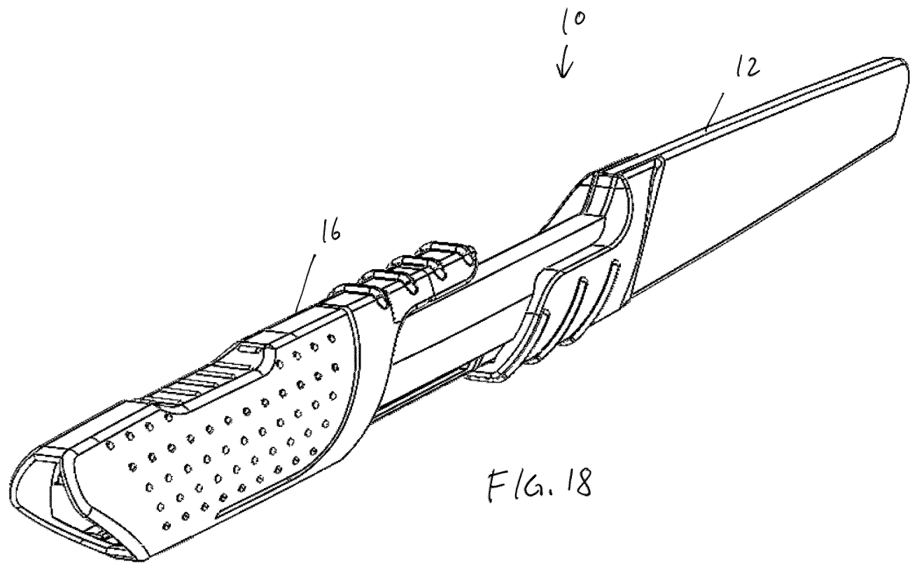

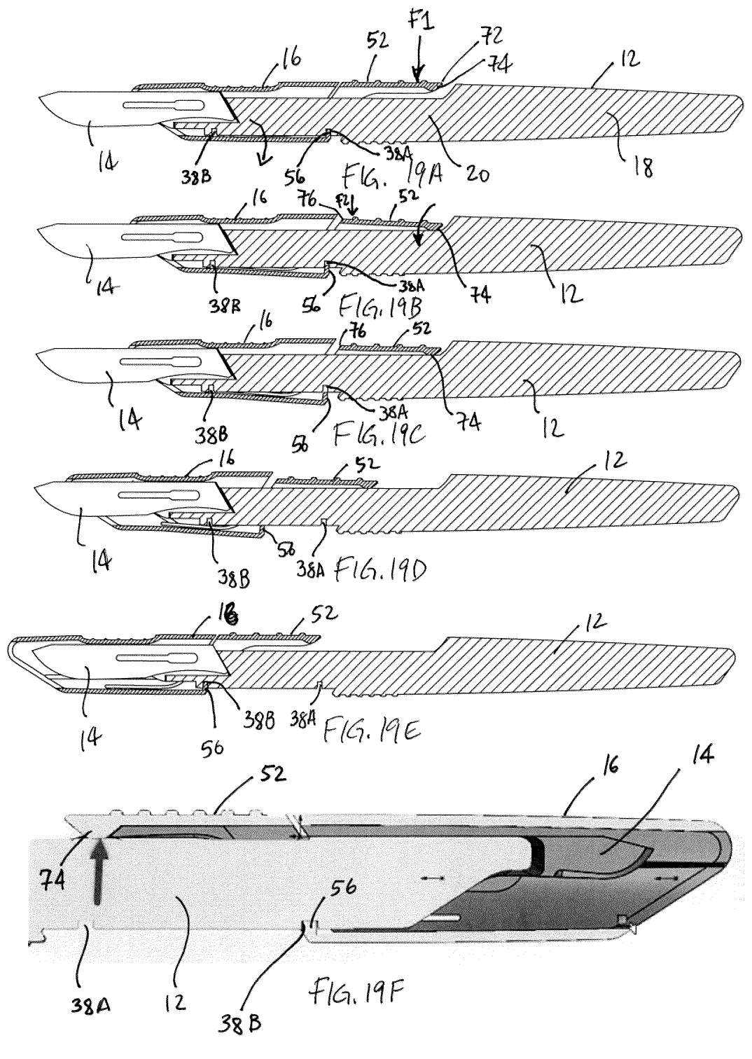

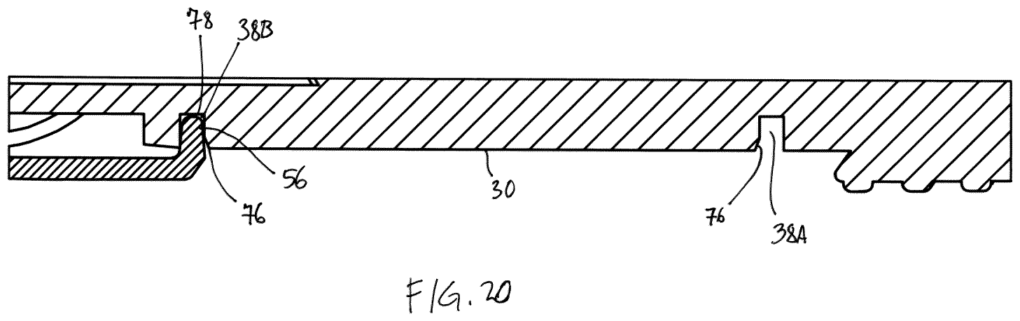

The shield 16 is movably mounted to the handle 12, particularly to the distal portion 20, using any arrangement. By way of non-limiting example, as shown in FIG. 14, the shield 16 may be provided with one or more inwardly-extending detents 54 formed to be received in the channels 32 for sliding movement therealong. The detents 54 may limit distal or proximal movement of the shield 16 relative to the handle 12. For example, engagement of at least one of the detents 54 with proximal end 35 (FIG. 10) of the respective channel 32 limits proximal movement of the shield 16 relative to the handle 12. As shown in FIGS. 21-24, at least one channel 32 may be delimited by the proximal end 35 and a distal end 37 to define a range of motion of the shield 16 relative to the handle 12. The guide ribs 34 assist in maintaining the detents 54 in the channels 32 to limit separation therebetween. The rail 36, having an enlarged cross-section, may add lateral stability to the shield 16 in limiting transverse movement of the shield 16 about the distal portion 20. Optionally, one or more sets of complementary ridges and channels may be provided on the shield 16 and the handle 12 to provide stability along axial movement. The ridge(s) and channel(s) may extend longitudinally with each of the ridge(s) being received in a corresponding channel. The length of each of the ridge(s) is less than the length of the corresponding channel to permit the ridge to translate along the corresponding channel.

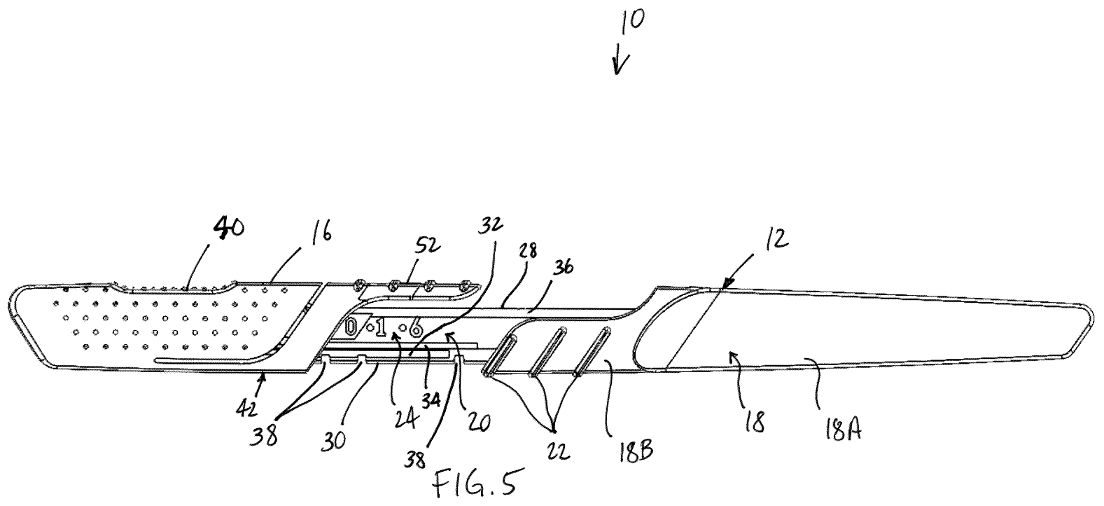

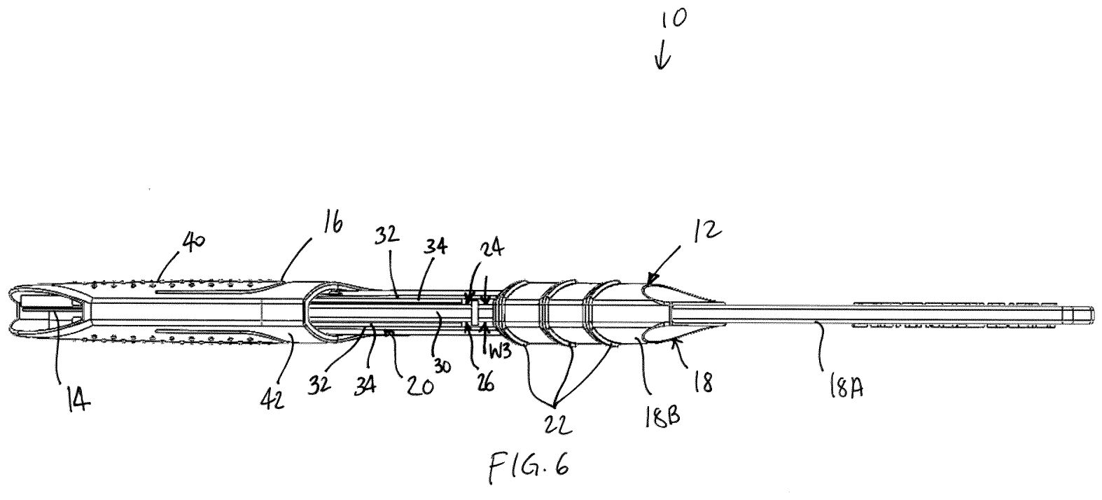

One or more tabs 56 are formed on the lever 42, e.g., on the cantilevered member 46, configured to be received within each of the notches 38. Preferably, with the lever 42 being in an unbiased (undisplaced) state, the tab 56 is snap received within the notches 38. The inherent memory of the pivotal connection 44 generates a restoring force which causes the tab 56 to be snap received. FIGS. 7-9 show the tab 56 being received in two different notches 38. With the tab 56 being received in one of the notches 38, movement of the shield 16 is inhibited. The tab 56 is freed from the corresponding notch 38 by depressing the lever 42 to cause displacement thereof, thereby removing the tab 56 from the corresponding notch 38. With the tab 56 removed from the corresponding notch 38, the shield 16 is released and free to move along the handle 12. With release of the displacing force against the lever 42, and movement of the shield 16 away from the corresponding notch 38, the restoring force of the pivotal connection causes the tab 56 to press against the lower edge 30. The shield 16 may be advanced to a different notch 38 where the tab 56 is snap received under bias of the restoring force.

The notches 38 are positioned to maintain the shield 16 in various positions. For example, a first of the notches 38A is positioned to locate and maintain the shield 16 in the first, use position with the blade 14 being exposed. In the first position, the shield 16 may fully expose the blade 14 or partially expose the blade 14 with a working length of the blade 14 being exposed. A second of the notches 38B is in position to locate and maintain the shield 16 in the second, post-use position with the blade 14 being fully covered (i.e., the shield 16 extends distally past the blade 14). In the second position, the shield 16 limits contact with the blade 14.

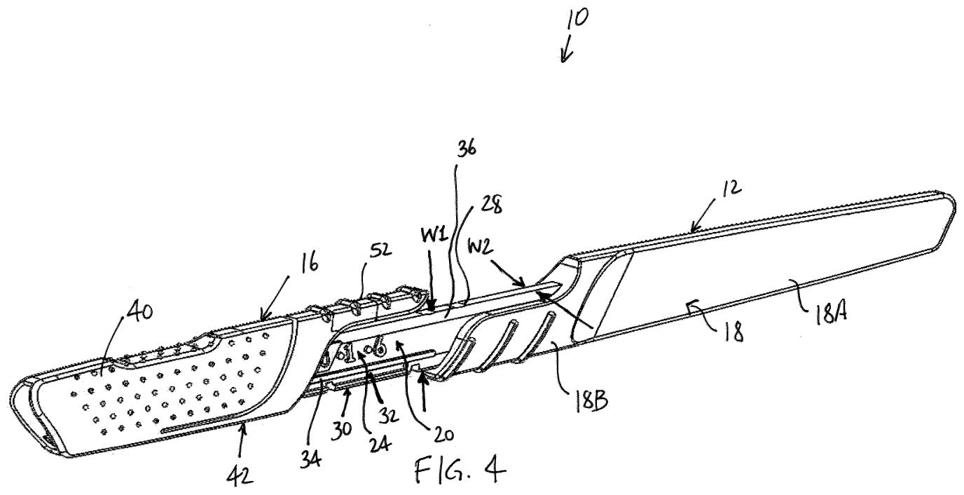

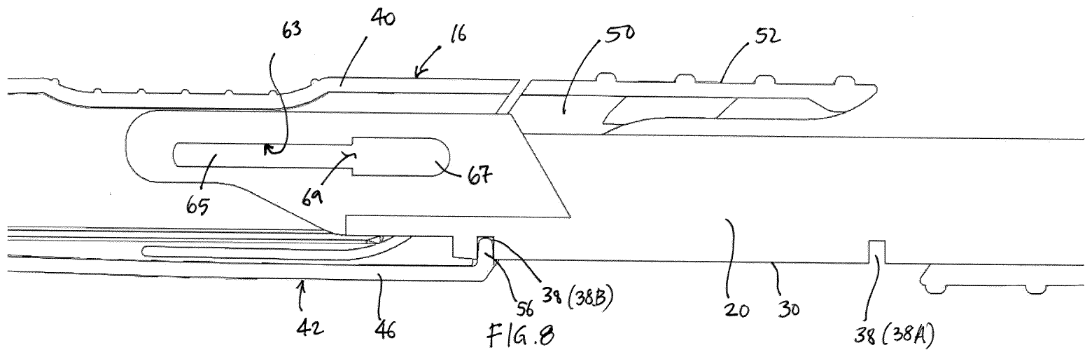

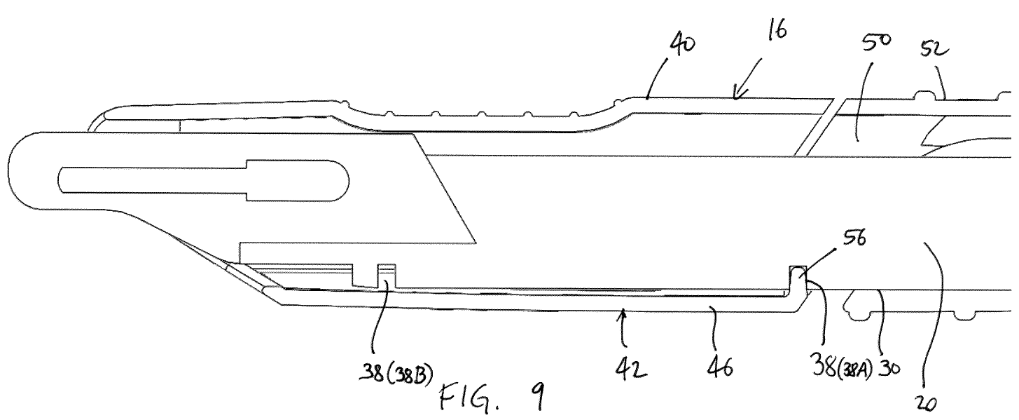

In addition, as shown in FIG. 5, one or more additional intermediate notches 38 may be provided to locate and maintain the shield 16 in positions with limited exposure of the blade 14. For example, FIGS. 10-11 show a third position, corresponding to a third notch 38, with very minimal exposure of the blade 14, whereas, FIGS. 12-13 show a fourth position, corresponding to a fourth notch 38, with more blade exposure than the third position but less than that provided by the first position. By way of non-limiting example, four of the notches 38 may be provided to control length of blade exposure of: 0 mm (fully shielded, post-use position), 1 mm (third position), 6 mm (fourth position), and working length (first position). The quantities and axial positions of the notches 38 may be varied depending on the length of the blade 14, the shape of the cutting edge of the blade 14 and intended use (procedure) of the surgical safety knife 10.

To assist in moving the shield 16, friction-enhancing features 58 may be provided on external portions of the shield 16, such as ridges, raised dots, indentations, knurled surfacing, textured surfacing, and so forth. One or more indentations 60 may be also provided along the spine 62 of the shield 16 formed to receive a forefinger of a user.

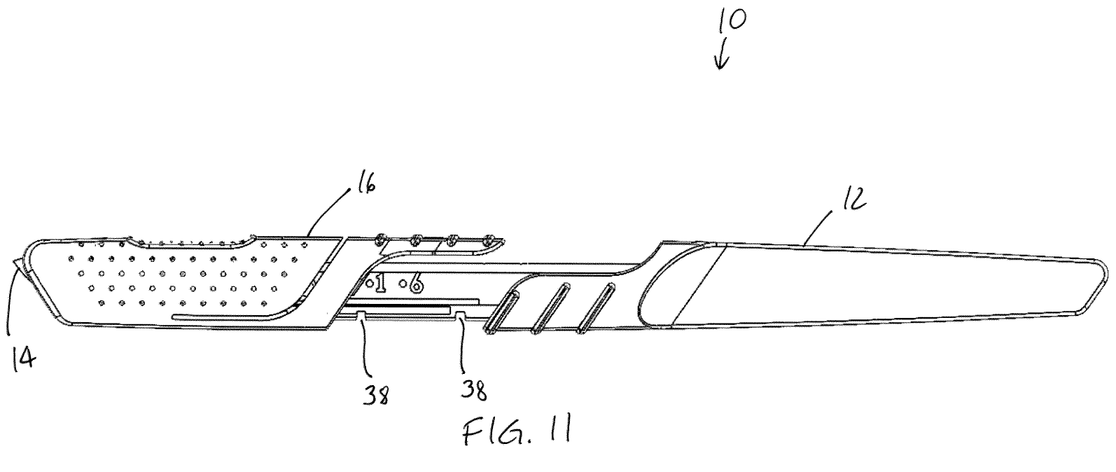

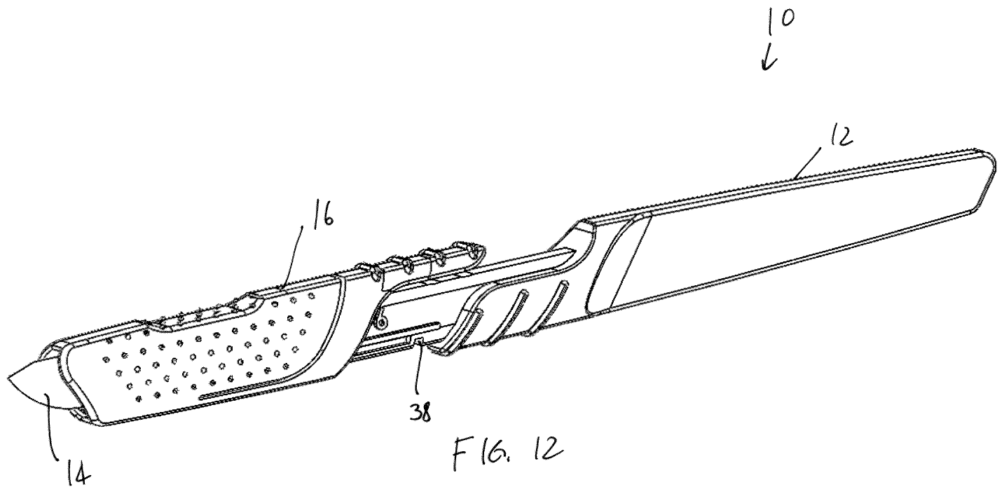

The blade 14 may be any known blade useable in surgery, autopsies, or other medical or scientific applications. The blade 16 is mounted to the distal portion 20 using any known technique, such as adhesion, over-molding, and so forth. The blade 14 may include a mounting opening 63 having a distal first portion 65 and an enlarged proximal second portion 67. A mounting block 69 may be provided on the distal portion 20 formed to interferingly fit in the mounting opening 63 to have the blade 14 retained on the distal portion 20. A guide block 71 may be provided to protrude from the distal portion 20 opposite the blade 14. As shown in FIGS. 3 and 14, the guide block 71 may be formed to extend into close proximity with an interior surface 73 of the shield 16 to limit transverse displacement of the blade 14, particularly during use, in providing stability thereto. The blade 14 may be of any useable material (e.g., being metallic, ceramic, thermoplastic, and combinations thereof) and include one or more cutting edges 64 configured in any manner. As shown in FIG. 1, the cutting edge 64 may be rounded, or, as shown in FIGS. 15-16, the cutting edge 64 may be straight.

The shield 16 is preferably formed with sufficient length to extend distally past the blade 14 with the shield 16 in the second, post-use position. The shield 16 may have a distal end 66 which is tapered, as shown in FIG. 1. In addition, junction 68 defined at the intersection of the spine 62 and the distal end 66 may be rounded or truncated to provide a minimally-obstructed sight line to a user over the shield 16 to the blade 14.

The shield 16 and the distal portion 20 may include cooperating locking members to lock the shield 16 in the second, post-use position. The shield 16 may be reversibly movable along the length of the distal portion 20 outside of engagement with any of the notches 38. Locking members may be provided such that, with the tab 56 being received in the second notch 32B, the locking members engage to maintain the shield 16 in the second position, even with displacement of the lever 42. The cooperating locking members are provided separate from any of the notches 38 and the tab 56, thus accordingly, the lever 42 does not release the shield 16 from the cooperating locking members. The cooperating locking members may be complementary ramped structures, or the like, formed on the shield 16 and the distal portion 20, which permit unidirectional by-pass movement with resistance to reverse movement. In this manner, the shield 16 may be advanced distally to the post-use, shielding position, but, thereafter, is inhibited from moving in reverse, proximally, to expose the blade 14.

The shield 16 advantageously is separate from the proximal portion 18 of the handle 12, with the two pieces coacting as a grip for a user. Moreover, the surgical safety knife 10 may be provided to a point-of-use, e.g., a surgical location, with the shield 16 in the first position. From the first position, the shield 16 may be manipulated with one hand of a user with depression of the lever 42 to allow movement of the shield 16 to the other positions. As shown in FIG. 1, the distal end of the shield 16 may be formed with a shape complementary to a proximal end of the proximal portion 18. In addition, the shield 16 may have an oval profile (FIG. 14) matching that of the forward portion 18A of the proximal portion 18. Thus, as shown in FIG. 1, in a use position, the shield 16 may form a relative continuous handling surface with the proximal portion 18. A discontinuity 70 is formed between the shield 16 and the proximal portion 18. The discontinuity 70 allows the lever 42 to be displaced as discussed above. The lever 42 is part of the grip area of the surgical safety knife 10 and provides a depressible mode of releasing the shield 16 without disrupting the grip area.

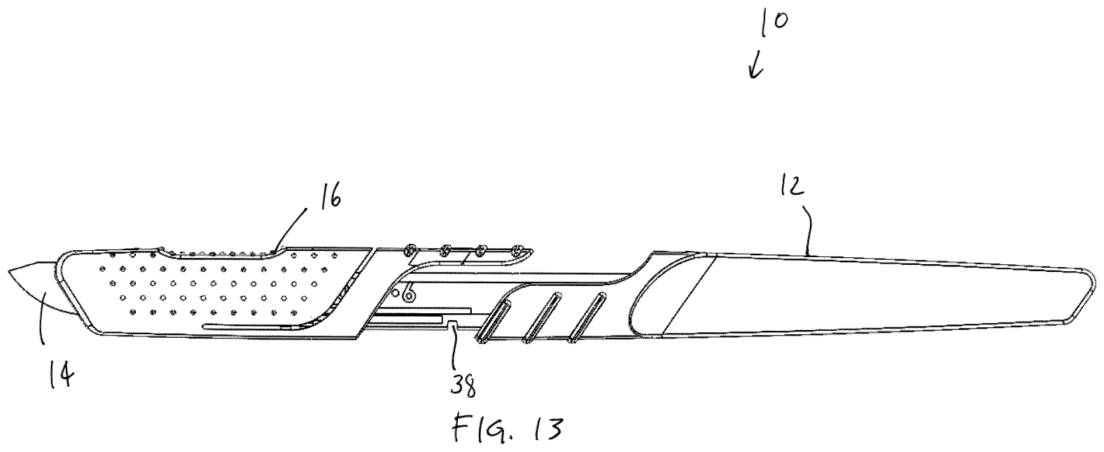

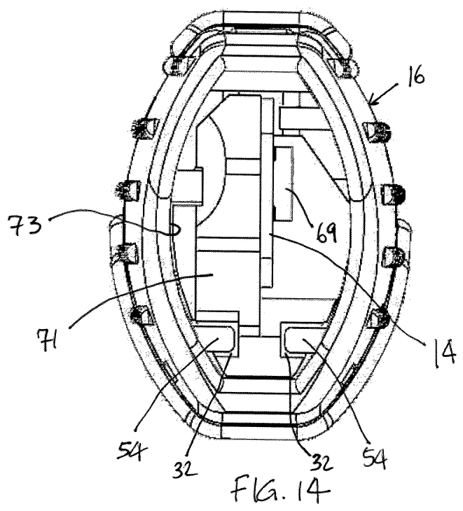

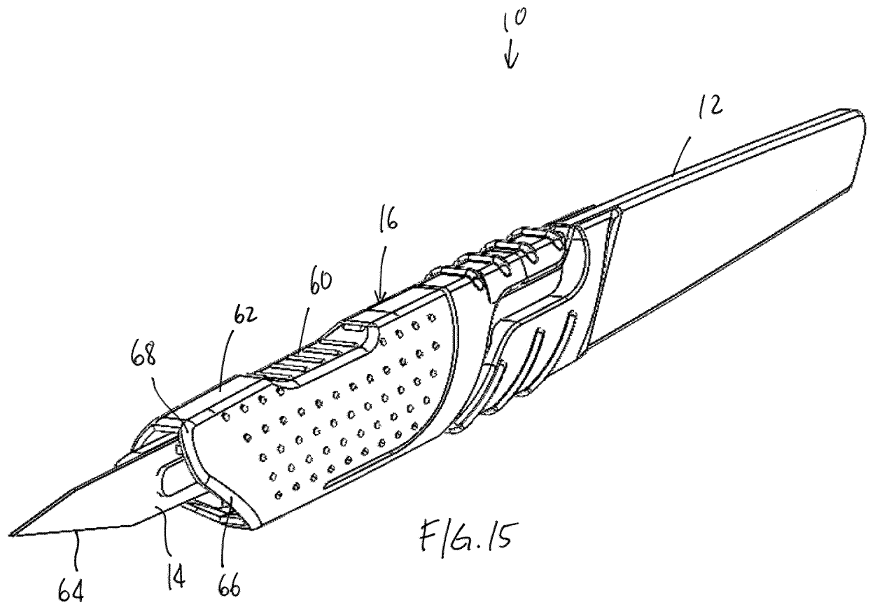

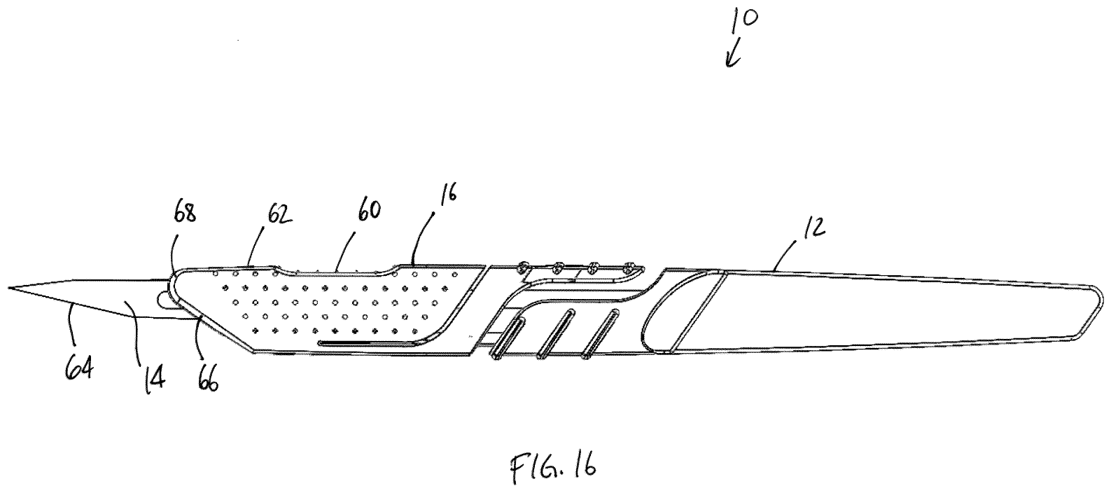

With reference to FIGS. 19A-19E, in a further embodiment, the surgical safety knife 10 may be provided to require a two-step release. As discussed above, the tab 56 is releasable from the any of the notches 38 with displacement of the lever 42. This, however, may result in inadvertent release of the shield 16 prior to use, e.g., during transportation or storage. The tail 52 can be modified to require two separate applications of torque to release the tab 56. It is preferred that the surgical safety knife 10 be provided, prior to use, with the shield 16 in the first, use position, e.g. with the tab 56 seated in the first notch 38A. With reference to FIGS. 19A-19B, proximal end 72 of the tail 52 may be provided with a resting tab 74, on an interior surface thereof. The resting tab 74 may be formed with an arcuate or hemi-spherical shape. In a first step to release the shield 16, as shown in FIG. 19A, a first force F1 is applied to the tail 52 to cause the resting tab 74 to come into pressing contact with the handle 12, e.g., into pressing contact with the distal portion 20. The first force F1 creates a first moment about the pivotal connection 44. The handle 12 limits the displacement of the tail 52, as shown in FIG. 19B. This displacement is limited so that the tab 56 is not moved clear of the first notch 38A. Accordingly, this arrangement prevents the shield 16 from being released with a single force applied to the tail 52.

To release the shield 16, in a second step, as shown in FIG. 19B, a second force F2 is applied to the tail 52, distally of the resting tab 74, to create a moment about the resting tab 74. This results in downward displacement of distal end 76 of the tail 52, as shown in FIG. 19C. This secondary displacement further drives the lever 42 downwardly resulting in further displacement of the tab 56 with the tab 56 clearing the first notch 38A. With the tab 56 clear of the notch 38A, the shield 16 is distally advanceable, as shown in FIG. 19D. With release of the tail 52, the lever 42 is urged to return to its undisplaced state. With sufficient advancement of the shield 16, the tab 56 is snap received in the second notch 38B, with the shield 16 covering the blade 14.

As shown in FIG. 19F, in an alternate configuration, the resting tab 74 may be configured to be in continuous contact with the handle 12. With this configuration, as shown by the arrow, a normal force is continually applied against the resting tab 74, resulting in a continuous tension being generated in the tail 52 about the lever 42, which urges the tab 56 towards the handle 12. The continuous contact between the resting tab 74 and the handle 12 may be generated by interfering engagement therebetween. With this arrangement, the first force F1 discussed above is not required to urge the resting tab 74 into engagement with the handle. The second force F2 is applied in similar manner as discussed above in connection with FIG. 19B to displace the tab 56. With the configuration of FIG. 19F, a one-step release is required.

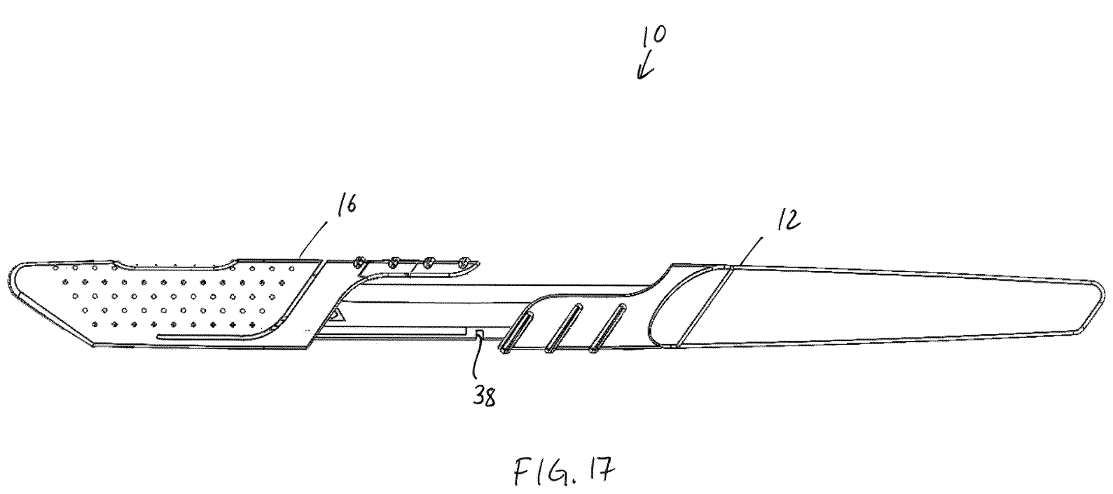

As a further variation, as shown in FIG. 20, one or the more of the notches 38 may be provided with a chamfered or truncated edge 76 on the side corresponding to desired direction of movement of the shield 16. For example, with distal movement intended for the shield 16, for the first notch 38A, the chamfered edge 76 may be located on its distal edge, while, with the second notch 38B, the chamfered edge 76 may be located on its proximal edge. In this manner, the tab 56 is assisted in separating from the first notch 38A to move distally, and the tab 56 is assisted in seating into the second notch 38B upon reaching the second notch 38B. Free end 78 of the tab 56 may be rounded to further facilitate the tab 56 passing over the chamfered edge(s) 76. In addition, edges of the notches 38 may be provided as right angles to best resist the by-passing of the tab 56, in limiting movement of the shield 16. Thus, with distal movement intended for the shield 16, the proximal edge of first notch 38A may be provided as a right angle. To limit distal movement of the shield 16 to the second notch 38B, the distal edge of the second notch 38B may be provided as a right angle.

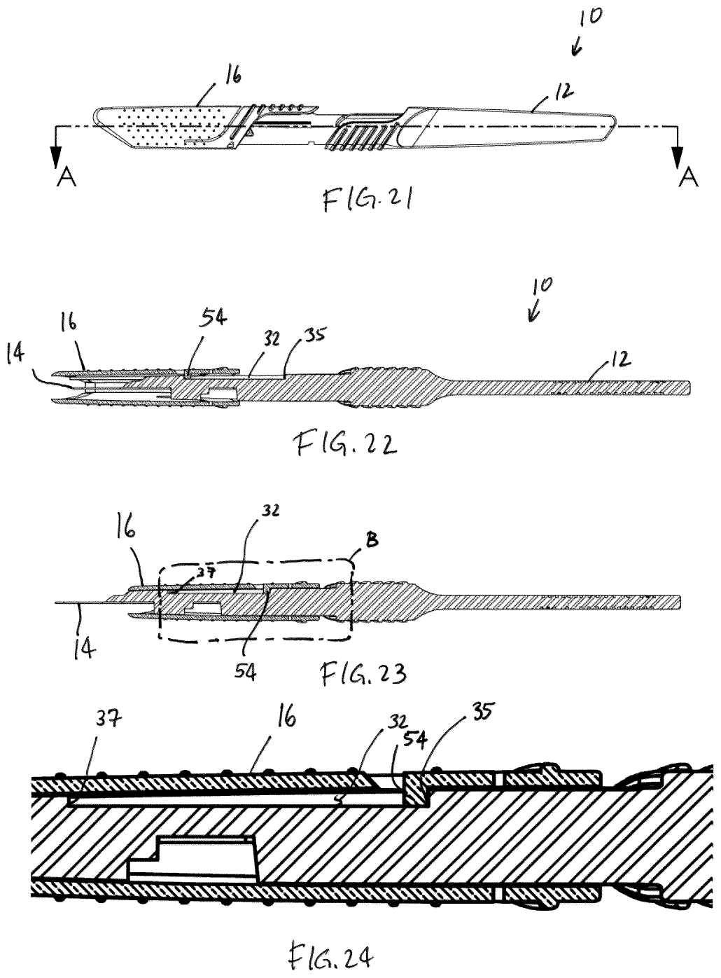

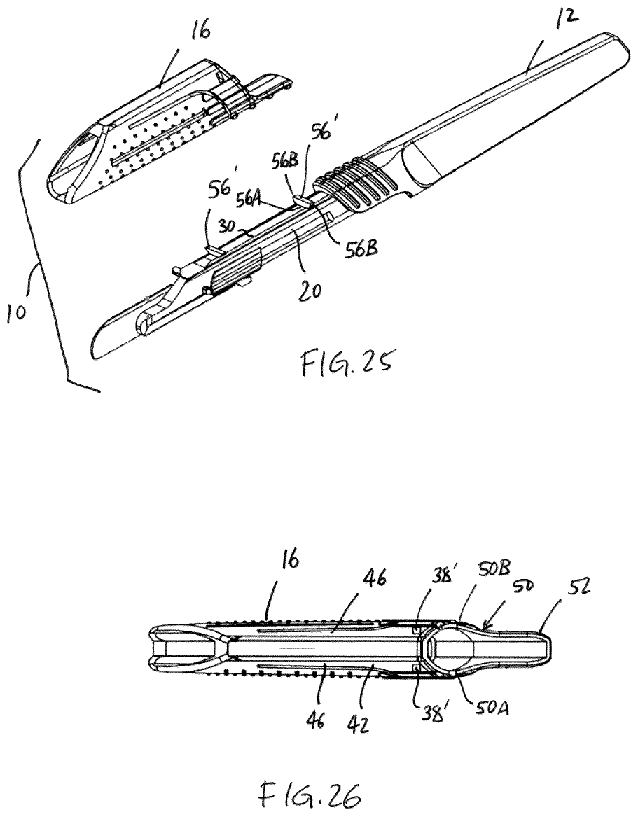

In addition, as shown in FIGS. 25-33, the notches 38 on the distal portion 20 may be replaced with tabs 56′, with the tabs 56 on the shield 16 being replaced with one or more notches 38′. This allows for the same operation as described above, but with the locations of the elements being reversed between the shield 16 and the distal portion 20. Here, displacement of the lever 42 causes the one or more notches 38′ to move away from the distal portion 20 in separating from an engaged tab 56′. With reference to FIG. 25-28, the tabs 56′ may be each formed elongated with a connecting base 56A, extending from the distal portion 20 (e.g., extending from the lower edge 30), with one or more tab wings 56B extending from the connecting base 56A transversely to a longitudinal axis of the distal portion 20. Correspondingly, as shown in FIG. 26, the notches 38′ may be formed on the lever 42, preferably, in general axial alignment with the cantilevered member 46, so that, with the lever 42 in an undisplaced state, an engaged tab 56′ may be seated in at least one of the notches 38′, as shown in FIGS. 27-28. It is preferred that a pair of notches 38′ be provided on opposing sides of the yoke 50. The notches 38′ may be positioned to simultaneously receive both of the tab wings 56B of a respective tab 56′. In addition, as shown in FIG. 26, it is preferred that the shield 16 have an oval profile with legs 50A, 50B of the yoke 50 being outwardly bowed in extending away from the cantilevered member 46, towards the tail 52. This provides clearance for an engaged tab 56′ to be disengaged from the notches 38′ with displacement of the lever 42. Return of the lever 42 to the undisplaced state allows the tab 56′ to be snap received in the notches 38′, particularly with the tab wings 56B being received in the notches 38′. As will be appreciated by those skilled in the art, the tabs 56′ are positioned, spaced apart, on the distal portion 20 to correspond to the different positions of the shield 16 (first (use) position, second (shielded) position, and so forth). The notches 38′ may be through-holes extending through the lever 42 or interiorly-located indentations.

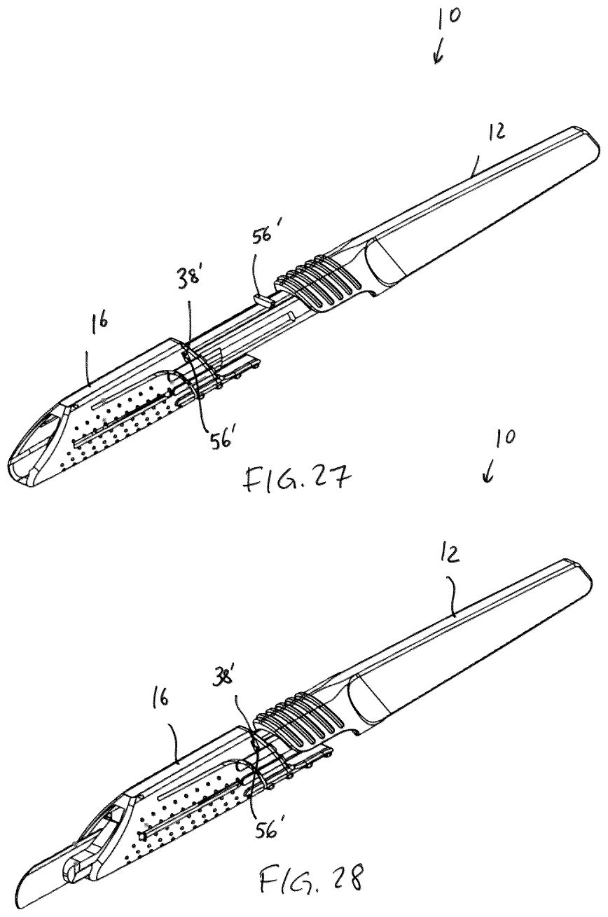

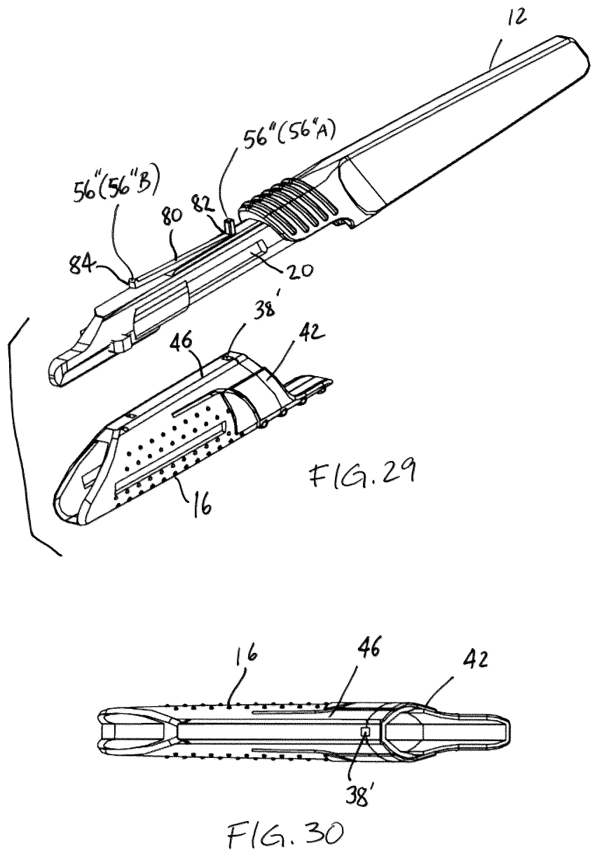

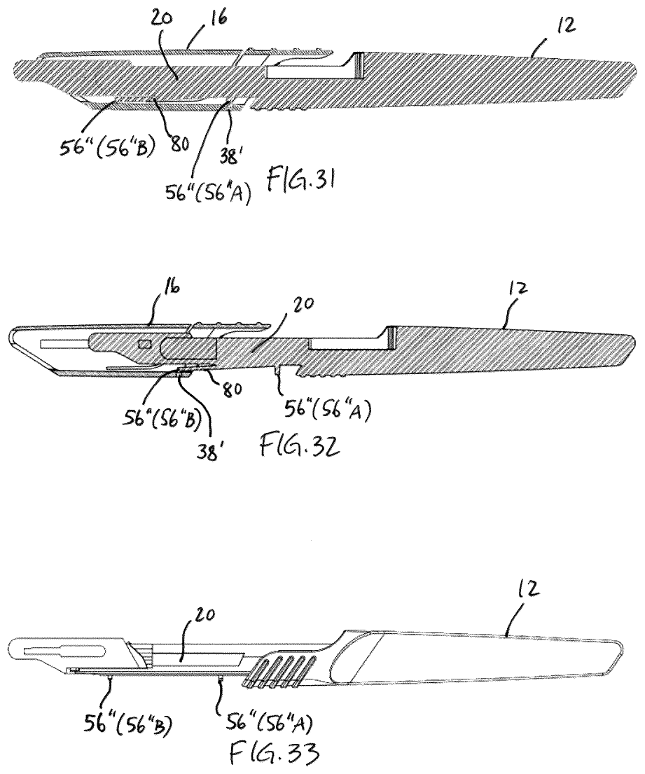

With reference to FIGS. 29-33, the tabs 56′ may be pin shaped (56″), extending from the distal portion 20 (e.g., from the lower edge 30), to be snap received in corresponding notch 38′ formed on the lever 42. One notch 38′ may be provided with the tabs 56″ being pin shaped, the notch 38′ being located on an interior of the cantilevered member 46, as shown in FIG. 30. Displacement of the lever 42 causes the tabs 56″ to be freed from the notch 38′. Restoring force of the lever 42, urging the lever 42 to the undisplaced state, causes one of the tabs 56″ to be snap received in the notch 38′ and maintained therein. One of the tabs 56″ (56″A) may be provided as a rigid structure, preferably coinciding with a start position of the shield 16 (e.g., corresponding to the first, use position as shown in FIG. 31). Further tabs 56″ (e.g., 56″B) may be formed to be deflectable, such as individually being located on a deflectable arm 80. The deflectable arm 80 may be secured at a proximal end 82 to the distal portion 20 from which it extends distally to a free distal end 84. In an unstressed condition, as shown in FIG. 29, the free distal end 84 is spaced from the distal portion 20. The deflectable arm 80 should be formed with inherent memory to be urged to the unstressed condition (e.g., in the same manner as described above with respect to the pivotal connection 44). The deflectable arm 80 may be formed as a V-shaped spring clip (metallic, polymeric, or a combination thereof) with one leg of the spring clip being attached along the distal portion 20 with the vertex of the spring clip coinciding with the proximal end 82. The tab 56″B may be located on, or near, the free distal end 84. With this arrangement, as the shield 16 is advanced distally from the use position, the deflectable arm 80 may be deflected inwardly towards the distal portion 20. With sufficient distal advancement, the tab 56″B snap engages the notch 38′ located on the lever 42, as shown in FIG. 32. Restoring force generated by the inherent memory in the deflectable arm 80, and/or by the inherent memory of the lever 42, urges the tab 56″B into snap engagement with the notch 38′ and maintains engagement therewith. Further displacement of the lever 42 may cause the tab 56″B to disengage from the notch 38′. As will be appreciated by those skilled in the art, the tabs 56″ are positioned, spaced apart, on the distal portion 20 to correspond to the different positions of the shield 16 (first (use) position, second (shielded) position, and so forth). The notch 38′ may be formed slightly larger than the tabs 56″ (56″A, 56″B). The notch 38′ may be a through-hole in the lever 42 or an interiorly-located indentation. As shown in FIG. 33, the tab 56″B may be provided as a rigid structure, in the same manner as the tab 56″A.

As non-limiting examples, a primary grip zone of the surgical safety knife may be defined between 20 mm and 75 mm from a distal end of the distal portion 20. Within the noted range, all or a portion of the shield 16 may be part of the primary grip zone, with the handle 12 making up the difference. At least 80% of the length of the primary grip zone may have an oval profile as seen in FIG. 14, and a majority of the primary grip zone may be provided with friction-enhanced features, as described above. The tail 52 may extend proximally beyond the primary grip zone. The width of the primary grip zone may be between 5 mm and 15 mm, with the height of the profile, e.g. between flat top to flat bottom, being within the range of 7 mm to 20 mm.

In addition, the center of mass of the surgical safety knife 10, without the blade 14, may be located between 20 mm and 75 mm from a distal end of the distal portion 20.

What is claimed is:

1. A surgical safety knife comprising:
an elongated handle having a proximal portion, defining a grip for a user, and a distal portion, having a reduced cross-section compared to the proximal portion, wherein, the distal portion is elongated with first and second notches being formed thereon, spaced apart along the length of the distal portion;
a blade extending distally from the distal portion of the handle; and,
a shield moveably mounted to the distal portion of the handle so as to be moveable along the handle between a first position, where the blade is exposed, to a second position, where the shield fully covers the blade, wherein, the shield includes a main body and a lever pivotally coupled to the main body, the main body being in contact with the distal portion of the handle, the lever being pivotally connected to the main body so as to be pivotally displaceable relative to the main body between an undisplaced state and a displaced state, wherein, a tab is provided on the lever formed to be selectively snap received within the first notch and the second notch with the lever being in the undisplaced state, wherein, with the tab being received in the first notch, the shield is retained in the first position, and, wherein, displacement of the lever to the displaced state results in the tab being freed from the first notch to release the shield and to allow the shield to move along the handle from the first position.

2. The surgical safety knife of claim 1, wherein the first notch and the second notch are formed on a common face of the distal portion.

3. The surgical safety knife of claim 2, wherein, the distal portion has a generally rectangular cross-section with opposing upper and lower edges, and, wherein the lower edge is the common face.

4. The surgical safety knife of claim 1, wherein, the pivotal connection includes inherent memory which urges the lever to return to the undisplaced state.

5. The surgical safety knife of claim 1, wherein, a third notch is formed on the distal portion located between, and spaced from both, the first and second notches, the third notch being formed to snap receive the tab with the lever being in the undisplaced state, and, wherein, with the tab received in the third notch, the shield is retained in a third position with the blade being exposed less than the amount of the blade is exposed in the first position.

6. The surgical safety knife of claim 5, wherein a fourth notch is formed on the distal portion located between, and spaced from both, the second and third notches, the fourth notch being formed to snap receive the tab with the lever being in the undisplaced state, and, wherein, with the tab received in the fourth notch, the shield is retained in a fourth position with the blade being exposed less than the amount of the blade is exposed in the third position.

7. The surgical safety knife of claim 1, wherein, the shield and the distal portion include cooperating locking members to lock the shield in the second position.

8. The surgical safety knife of claim 7, wherein, the cooperating locking members are spaced from the tab.

9. The surgical safety knife of claim 1, wherein, the tab is snap received within the second notch with movement of the shield to the second position and with the lever in the undisplaced state.

10. The surgical safety knife of claim 1, wherein, the shield includes friction-enhancing features on external portions thereof.

11. The surgical safety knife of claim 1, wherein, the lever is displaced by two forces, applied in sequence.

12. The surgical safety knife of claim 11, wherein, a first of the two forces is applied to a protruding tail of the lever to cause a proximal end of the tail to be displaced.

13. The surgical safety knife of claim 12, wherein, a second of the two forces is applied to the tail to generate a moment about the displaced proximal end to displace a distal end of the tail.

14. A surgical safety knife comprising:
an elongated handle having a proximal portion, defining a grip for a user, and a distal portion, having a reduced cross-section compared to the proximal portion, wherein, the distal portion is elongated with first and second tabs protruding therefrom, spaced apart along the length of the distal portion;
a blade extending distally from the distal portion of the handle; and,
a shield moveably mounted to the distal portion of the handle so as to be moveable along the handle between a first position, where the blade is exposed, to a second position, where the shield fully covers the blade, wherein, the shield includes a main body and a lever pivotally coupled to the main body, the main body being in contact with the distal portion of the handle, the lever being pivotally connected to the main body so as to be pivotally displaceable relative to the main body between an undisplaced state and a displaced state, wherein, at least one notch is provided on the lever formed to selectively snap receive one of the first tab and the second tab with the lever being in the undisplaced state, wherein, with the first tab being received in the at least one notch, the shield is retained in the first position, and, wherein, displacement of the lever to the displaced state results in the first tab being freed from the at least one notch to release the shield and to allow the shield to move along the handle from the first position.

15. The surgical safety knife of claim 14, wherein the first tab includes a connecting base, extending from the distal portion, and one or more tab wings extending from the connecting base transversely to a longitudinal axis of the distal portion.

16. The surgical safety knife of claim 15, wherein two of the tab wings are formed on the first tab, and, wherein the at least one notch includes two notches positioned on the lever to simultaneously receive the two of the tab wings.

17. The surgical safety knife of claim 14, wherein the first tab and the second tab are each pin shaped.

18. The surgical safety knife of claim 17, wherein the first tab is a rigid structure.

19. The surgical safety knife of claim 18, wherein the second tab is located on, or near, a free distal end of a deflectable arm fixed to the distal portion.

20. The surgical safety knife of claim 18, wherein the second tab is a rigid structure.
